SEQUENCE LISTING (2 sequences)

SEQ ID NO: 1 - DNA, 20 bp, synthetic construct
  source 1..20: mol_type = other DNA
tctacatcacgccgttccag

SEQ ID NO: 2 - DNA, 20 bp, synthetic construct
  source 1..20: mol_type = other DNA
ccactccattccaagctttc

(19) United States
(12) Patent Application Publication
(10) Pub. No.: US 2025/0346966 A1
(43) Pub. Date: Nov. 13, 2025

(54) PRIMER FOR AMPLIFYING CLOSELY LINKED MOLECULAR MARKER OF POWDERY MILDEW RESISTANCE GENE PmDR803 OF TRITICUM CARTHLICUM AND APPLICATION THEREOF

(71) Applicants: Crop Research Institute, Shandong Academy of Agricultural Sciences, Jinan (CN); Yantai University, Yantai (CN); National Center of Technology Innovation for Comprehensive Utilization of Saline-Alkali Land, Dongying (CN)

(72) Inventors: Cheng LIU, Jinan (CN); Pengtao MA, Yantai (CN); Yuli JIN, Yantai (CN); Kai WANG, Jinan (CN); Guang QI, Jinan (CN); Xiaolu WANG, Jinan (CN); Jun GUO, Jinan (CN); Dungong CHENG, Jinan (CN); Yan ZI, Jinan (CN); Jianjun LIU, Jinan (CN)

(21) Appl. No.: 19/016,024

(22) Filed: Jan. 10, 2025

(30) Foreign Application Priority Data
May 8, 2024 (CN) 2024105612421

(51) Int. Cl. C12Q 1/6895

(52) U.S. Cl. CPC C12Q 1/6895; C12Q 2600/13; C12Q 2600/156

(57) ABSTRACT

Disclosed is a primer for amplifying a closely linked molecular marker of a powdery mildew resistance gene PmDR803 of Triticum carthlicum. The molecular marker primer includes a forward primer HENU629-F and a reverse primer HENU629-R, where the nucleotide sequence of the forward primer HENU629-F is shown as SEQ ID NO:1, and the nucleotide sequence of the reverse primer HENU629-R is shown as SEQ ID NO:2. The molecular marker primer provided by the present disclosure can be applied in detection and identification of the gene PmDR803, auxiliary identification of a powdery mildew resistance trait of wheat, and molecular marker-assisted selection breeding.

### TECHNICAL FIELD

The present disclosure relates to the technical field of agricultural biological genes, and in particular to a primer for amplifying a closely linked molecular marker of a powdery mildew resistance gene PmDR803 of Triticum carthlicum and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25002TLAN-USP1-SL.xml. The XML file is 2,928 bytes; is created on Jan. 9, 2025; and is being submitted electronically via patent center.

### BACKGROUND

Wheat is an important food crop in the world, is called the three major food crops together with rice and corn, and provides energy intake for 35-40% of the world population (Lu et al. World wheat industry development and trend analysis in 2017. China Market, 2017, 26:63-65). Wheat powdery mildew is one of the three major fungal diseases of wheat, which has the characteristics of a wide spread range, a fast spread speed and serious damage, resulting in reduction of a wheat yield by 5-10%, and serious yield reduction by 50% (Liu et al. Analysis of causes and trends of occurrence succession of wheat powdery mildew in China. Plant Protection, 1991, 6:7-8). Measures to control the wheat powdery mildew mainly include chemical control and breeding and popularization of resistance varieties. Compared with the traditional chemical control method, the method of digging wheat varieties with resistance genes and breeding disease-resistant varieties by using resistance sources and resistance genes is a safe, economic and effective method for controlling the wheat powdery mildew.

At present, 69 powdery mildew resistance genes (Pm1-Pm69) of wheat are officially named, including 6 genetic loci, namely Pm3=Pm8=Pm17, Pm18=Pm1c, Pm22=Pm1e, Pm23=Pm4c, Pm31=Pm21, and Pm46=Pm48 (Wang et al. Fighting wheat powdery mildew: from genes to fields. Theoretical and applied genetics, 2023, 136:27). The powdery mildew resistance genes Pm12, Pm13, Pm16, Pm20, Pm21, and Pm30 still show high resistance in production, but the agronomic traits of the materials carrying the genes Pm12, Pm13, and Pm16 are poor, so the materials are not suitable for serving as breeding parents directly. (Huang et al. Identification of powdery mildew resistance genes in common wheat (Triticum aestivum L. em Thell). IX. Cultivals, land races and breeding lines grown in china. Plant Breeding, 1997, 116:223-238). Therefore, it is of great significance to continuously find new powdery mildew resistance genes for rational utilization of resistance sources, acceleration of gene polymerization breeding and broadening of genetic basis of wheat.

Rich excellent genes are contained in wheat relative species, and these genes have important utilization value for the genetic improvement of the wheat. These resources can not only be used for wheat genetic breeding, but also greatly broaden the genetic basis of the wheat, providing strong support for the improvement of genetic diversity, disease resistance and other aspects. Triticum carthlicum (AABB) is produced by crossing bread wheat with tetraploid wheat near the Altai Mountains in the process of spreading (Zhao et al. Population genomics unravels the Holocene history of bread wheat and its relatives. Nature Plants, 2023, 9:403-419). The Triticum carthlicum belongs to the secondary gene pool of common wheat. Up to now, Pm4b and Pm33 have been excavated from the Triticum carthlicum. The Triticum carthlicum is an important genetic resource for genetic improvement of the common wheat, which has excellent characteristics such as high resistance to powdery mildew and scattered smut, stem rust, low temperature resistance, pre-harvest sprouting resistance, strong tillering ability, good seed setting and a high protein content, and has great breeding potential. Therefore, it is very important to find and map new powdery mildew resistance genes from the Triticum carthlicum, develop molecular markers thereof and apply same to molecular marker-assisted selection breeding, so as to realize breeding of powdery mildew resistance varieties of wheat and effectively control the wheat powdery mildew.

### SUMMARY

An objective of the present disclosure is to provide a primer for amplifying a closely linked molecular marker of a powdery mildew resistance gene PmDR803 of Triticum carthlicum and an application thereof, so as to map and detect the powdery mildew resistance gene PmDR803 of Triticum carthlicum by utilizing the primer-amplified molecular marker, purposefully select parents during wheat breeding, and provide guidance basis for selection of powdery mildew-resistant new varieties of wheat.

The present disclosure is implemented through the following method: a primer for amplifying a closely linked molecular marker of a powdery mildew resistance gene PmDR803 of Triticum carthlicum, where the molecular marker primer includes a forward primer HENU629-F and a reverse primer HENU629-R.

A forward primer of a molecular marker HENU629 is HENU629-F, and the nucleotide sequence is:

5′-TCTACATCACGCCGTTCCAG-3′, as shown in SEQ
ID NO: 1.

A reverse primer of the molecular marker HENU629 is HENU629-R, and the nucleotide sequence is:

5′-CCACTCCATTCCAAGCTTTC-3′, as shown in SEQ
ID NO: 2.

The present disclosure provides an application of the primer for amplifying a closely linked molecular marker of a powdery mildew resistance gene PmDR803 of Triticum carthlicum in detection and identification of the gene PmDR803, auxiliary identification of a powdery mildew resistance trait of wheat, and molecular marker-assisted selection breeding.

The present disclosure further provides a method for detecting whether a wheat sample carries a powdery mildew resistance gene PmDR803 of Triticum carthlicum. The method includes the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product, where the molecular marker primer includes a forward primer HENU629-F and a reverse primer HENU629-R, the nucleotide sequence of the forward primer HENU629-F is shown as SEQ ID NO: 1, and the nucleotide sequence of the reverse primer HENU629-R is shown as SEQ ID NO: 2; and
(3) performing electrophoresis and detection on the amplification product, where if a specific band of 385 bp is capable of being amplified, the wheat sample to be detected is indicated to carry the powdery mildew resistance gene PmDR803 of Triticum carthlicum, otherwise, the wheat sample to be detected is indicated not to carry the powdery mildew resistance gene PmDR803 of Triticum carthlicum.

A PCR amplification system in step (2) is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification in step (2) is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.

A procedure of the electrophoresis of the amplification product in step (3) is: performing electrophoresis on a non-denaturing polyacrylamide gel with the mass volume percentage concentration of 8%, after the amplification product is mixed with 2 μL of 6× sample loading buffer, taking 2 μL of the mixture for spot sampling, performing electrophoresis under constant pressure of 180 V for 2.5-3 hours, and performing photographing after silver nitrate staining.

The Triticum carthlicum DR803 used in the research of the present disclosure shows high powdery mildew resistance in the seedling stage and an adult plant stage, and genetic analysis and molecular marker detection of powdery mildew resistance in the seedling stage show that the resistance of the Triticum carthlicum DR803 to wheat powdery mildew with different toxicity in the seedling stage is controlled by a pair of dominant genes PmDR803, which are mapped on a wheat chromosome 2 μL, and are a novel powdery mildew resistance gene/allele of wheat. Through genetic segregation population detection, the molecular marker HENU629 of the powdery mildew resistance gene PmDR803 of wheat provided by the present disclosure is found to have a genetic distance of 0.92 cM from the gene PmDR803, and be closely linked to the gene PmDR803, which can be used for accurately detecting the gene PmDR803 and a large genetic mapping population thereof, and can be effectively applied in identification and auxiliary identification of the powdery mildew resistance trait of wheat, molecular marker-assisted selection breeding, and fine mapping and map-based cloning of the gene PmDR803. The present disclosure provides the primer for amplifying a closely linked molecular marker of a powdery mildew resistance gene PmDR803 of wheat and the application thereof in wheat breeding of powdery mildew resistance, such that varieties carrying the gene PmDR803 are quickly and accurately screened, which is not affected by an environment, the selection target is clear, the production cost is saved, and the selection efficiency and quality of high-quality powdery mildew-resistant wheat varieties or strains are greatly improved.

### BRIEF DESCRIPTION OF THE DRAWING

FIG. 1 shows some results of detection of Triticum carthlicum DR803 and Triticum turgidum Langdon as well as segregation populations of hybrids thereof through a primer of a molecular marker HENU629.

In the FIGURE, M: DL2000; 1: Triticum carthlicum DR803 (powdery mildew resistance variety carrying the gene PmDR803); 2: Triticum turgidum Langdon (powdery mildew susceptible variety); 3-22: F_{2:3} families formed by crossing the Triticum carthlicum DR803 with the Triticum turgidum Langdon, where 3-7: F_{2:3} family homozygous for resistance, 8-17: F_{2:3} family for resistance and susceptibility segregation, and 18-22: F_{2:3} family homozygous for susceptibility; and the band indicated by the white arrow is a specific band capable of tracing the gene PmDR803.

### DETAILED DESCRIPTION OF THE EMBODIMENTS

The following embodiments are used for understanding the present disclosure better, but not intended to limit the present disclosure. Experimental methods in the following embodiments are conventional methods unless otherwise specified. All experimental materials, reagents, etc. used in the embodiments may be obtained from a commercial approach unless otherwise specified.

Embodiment 1 Development of molecular marker HENU629 primer of powdery mildew resistance gene PmDR803 of wheat

### 1. Materials

The resistance parent Triticum carthlicum DR803 and the susceptible parent Triticum turgidum Langdon are resistant and susceptible to wheat powdery mildew respectively. F₁ hybrids of the Triticum carthlicum DR803 and the Triticum turgidum Langdon are selfed to obtain a F₂ population and corresponding F_{2:3} families.

### 2. Genomic DNA Extraction of Wheat

A cetyltrimethylammonium bromide (CTAB) method for genomic DNA extraction of wheat includes the process as follows:
(1) Fresh wheat leaves are cut and ground with liquid nitrogen about 0.5 g of ground wheat leaves is put into a 2.0 mL centrifuge tube.
(2) 600 μL of CTAB extract solution is added and placed in a water bath at 65° C. for 40 minutes, where mixing up and down is performed once every 10 minutes.
(3) 600 μL of chloroform-isoamyl alcohol (24:1, v/v) is added and shaken gently on a shaking table for 15 minutes.
(4) Centrifugation is performed at 12000 rpm for 15 minutes, supernatant is taken to another 2 mL centrifuge tube, 3 times volume of precooled absolute ethanol is added, and precipitation is performed at a refrigerator at −20° C. for 60 minutes.
(5) Flocculent DNA precipitate is picked out and washed twice with 75% precooled ethanol.
(6) The DNA precipitate is picked out, placed in a 1.5 mL centrifuge tube, and air-dried indoors.
(7) 60 μL of TE buffer (100 mM Tris-Hcl, 10 mM EDTA, pH=8.0) is added to dissolve the DNA precipitate to prepare a DNA storage solution.
(8) The DNA storage solution is diluted to 20-30 ng/μL with ultrapure water as a working solution for later use.

### 3. Identification and Genetic Analysis of Powdery Mildew Resistance in Wheat Seedling Stage

The identification of powdery mildew resistance in a wheat seedling stage is completed in an artificial climate chamber. The resistance parent Triticum carthlicum DR803, the susceptible parent Triticum turgidum Langdon, the hybrid F₁, the F₂ population and the F_{2:3} families are planted in a 128-hole tray (2×2 cm), 5 seeds are sown in each hole, 20 seeds of each of the patents and F₁ are identified, 20 seeds of each F_{2:3} family are sown, and a susceptible control Mingxian 169 is sown randomly and labeled. After sowing, growth conditions are controlled as a 14 hours light/10 hours darkness cycle, a temperature of 20° C., and relative humidity of 30-40%. When the seedlings grow to a one leaf stage, and are inoculated with a wheat powdery mildew strain E09. The conditions for the first 24 hours after inoculation are controlled as follows: darkness, a temperature of 20° C., and relative humidity of 100%. Then, the conditions are controlled as follows: a 14 hours light/10 hours darkness cycle, a temperature of 18-22° C., and relative humidity of 100%. When the first leaf of the susceptible control Mingxian 169 is fully infected, phenotypes are recorded according to the standards of grades 0-4, where grades 0-2 are regarded as resistance grades, and grades 3 and 4 are regarded as susceptible grades.

Investigation results show that the Triticum carthlicum DR803 is immune to the strain E09, and the Triticum turgidum Langdon (LDN) is highly susceptible to the strain E09. Hybrid F₁ between the Triticum carthlicum DR803 and the Triticum turgidum Langdon (LDN) shows resistance. 105 of 138 F₂ plants show resistance, and 33 show susceptibility, which accords with a segregation ratio of resistance to susceptibility of 3:1. Among 138 F_{2:3} families, 32 are homozygous for resistance, 73 are heterozygous, 33 are homozygous for susceptibility, and the segregation ratio of homozygous resistance: heterozygosis: homozygous susceptibility is 1:2:1. Therefore, the resistance of the Triticum carthlicum DR803 to the strain E09 is controlled by a single dominant gene, named gene PmDR803.

### 4. Initial Mapping of Molecular Marker of Gene PmDR803

According to phenotypic identification results, 20 homozygous resistance families and 20 homozygous susceptible families are selected to construct a resistance pool and a susceptible pool respectively. Polymorphism detection is performed on the Triticum carthlicum DR803, the Triticum turgidum Langdon, the resistance pool and the susceptible pool by using genome-wide evenly distributed molecular markers. 10 pairs of markers show consistent polymorphism in the resistance and susceptible parents and the resistance and susceptible pools. Then, 138 F_{2:3} families of Triticum carthlicum DR803 xTriticum turgidum Langdon are genotyped by using these markers, and the gene PmDR803 is initially mapped at a terminal of the wheat chromosome 2 μL.

### 5. Development of Markers Closely Linked to Gene PmDR803

According to sequence information of the reference genome of Chinese spring wheat, simple sequence repeat (SSR) markers are designed through software primer5.0. The F_{2:3} families of Triticum carthlicum DR803×Triticum turgidum Langdon are genotyped, the SSR marker HENU629 closely linked to the gene PmDR803 is obtained, and a genetic distance is only 0.92 cM.

A primer of the molecular marker HENU629 includes a forward primer and a reverse primer:

nucleotide sequence of forward primer HENU629-F:
(shown in SEQ ID NO: 1)
5′-TCTACATCACGCCGTTCCAG-3′,
and
nucleotide sequence of reverse primer HENU629-R:
(shown in SEQ ID NO: 2)
5′-CCACTCCATTCCAAGCTTTC-3′.

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoresis of an amplification product is: electrophoresis is performed on a non-denaturing polyacrylamide gel with the mass volume percentage concentration of 8%, after the amplification product is mixed with 2 μL of 6× sample loading buffer, 2 μL of the mixture is taken for spot sampling, electrophoresis is performed under constant pressure of 180 V for 2.5-3 hours, and photographing is performed after silver nitrate staining.

Amplification results show: a specific bands of 385 bp is amplified, and wheat to be detected is indicated to carry the powdery mildew resistance gene PmDR803 of Triticum carthlicum; and the specific band of 385 bp fails to be amplified, and the wheat to be detected is indicated not to carry the powdery mildew resistance gene PmDR803 of Triticum carthlicum.

Embodiment 2 Application of molecular marker HENU629 primer of powdery mildew resistance gene PmDR803 of wheat

Triticum carthlicum DR803, Triticum turgidum Langdon and materials in F_{2:3} families formed by crossing the Triticum carthlicum DR803 with the Triticum turgidum Langdon are detected by the molecular marker HENU629 of the powdery mildew resistance gene PmDR803 of wheat, and a sample DNA extraction method is the same as that in Embodiment 1.

Samples to be detected: Triticum carthlicum DR803, Triticum turgidum Langdon and F_{2:3} families formed by crossing the Triticum carthlicum DR803 with the Triticum turgidum Langdon

Genomic DNAs of the above materials are extracted as a template of PCR amplification, and the amplification is performed through the primer of the molecular marker HENU629 developed by the present disclosure.

Nucleotide sequence of forward primer:
HENU629-F:
5′-TCTACATCACGCCGTTCCAG-3′,
and
nucleotide sequence of reverse primer:
HENU629-R:
5′-CCACTCCATTCCAAGCTTTC-3′.

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoretic segregation of an amplification product is: electrophoresis is performed on a non-denaturing polyacrylamide gel with the mass volume percentage concentration of 8%, after the amplification product is mixed with 2 μL of 6× sample loading buffer, 2 μL of the mixture is taken for spot sampling, electrophoresis is performed under constant pressure of 180 V for 2.5-3 hours, and photographing is performed after silver nitrate staining.

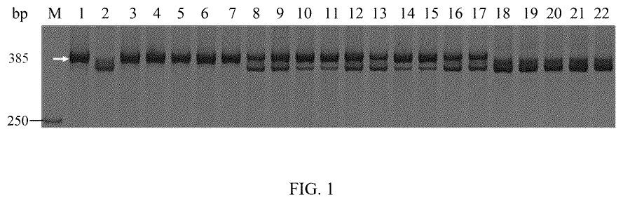

Detection results of the primer of the molecular marker HENU629 in the materials to be detected are shown in FIG. 1. FIG. 1 shows some amplification results of the primer of the molecular marker HENU629 in the F_{2:3} families derived from Triticum carthlicum DR803×Triticum turgidum Langdon. In the FIGURE, M: DL2000; 1: Triticum carthlicum DR803 (powdery mildew resistance variety carrying the gene PmDR803); 2: Triticum turgidum Langdon (powdery mildew susceptible variety); 3-22: F_{2:3} families formed by crossing the Triticum carthlicum DR803 with the Triticum turgidum Langdon, where 3-7: F_{2:3} family homozygous for resistance, 8-17: F_{2:3} family for resistance and susceptibility segregation, and 18-22: F_{2:3} family homozygous for susceptibility; and the band indicated by the white arrow is a specific band capable of tracing the gene PmDR803. Amplification results show that the primer of the marker HENU629 amplifies the specific band of 385 bp in the resistance parent Triticum carthlicum DR803 and the resistance families, and does not amplify the target band in the susceptible parent Triticum turgidum Langdon and the susceptible families.

The gene PmDR803 originated from the Triticum carthlicum DR803, and mapping and map-based cloning of the gene has not been reported yet. The molecular marker HENU629 and the primer provided by the present disclosure are used for detecting a large genetic mapping population, which contributes to fine mapping and map-based cloning of the gene PmDR803, and has very important significance for efficient transformation of the gene PmDR803 and in-depth analysis of a disease resistance mechanism.

The above embodiments are optimized implementation plans of the present disclosure, and are only used for illustrating the present disclosure, not limiting the present disclosure. All modifications or equivalent substitutions made by those skilled in the art without departing from the purpose and principle of the implementation plans of the present disclosure fall within the scope claimed to be protected by the present disclosure.

What is claimed is:

1. A primer for amplifying a closely linked molecular marker of a powdery mildew resistance gene PmDR803 of Triticum carthlicum, comprising a forward primer HENU629-F and a reverse primer HENU629-R, wherein the nucleotide sequence of the forward primer HENU629-F is shown as SEQ ID NO:1, and the nucleotide sequence of the reverse primer HENU629-R is shown as SEQ ID NO:2.

2. An application of the primer for amplifying a closely linked molecular marker of a powdery mildew resistance gene PmDR803 of Triticum carthlicum according to claim 1 in detection and identification of the gene PmDR803, auxiliary identification of a powdery mildew resistance trait of wheat, and molecular marker-assisted selection breeding.

3. A method for detecting whether a wheat sample carries a powdery mildew resistance gene PmDR803 of Triticum carthlicum, comprising the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product; and
(3) performing electrophoresis and detection on the amplification product, wherein if a specific band of 385 bp is capable of being amplified, the wheat sample to be detected is indicated to carry the powdery mildew resistance gene PmDR803 of Triticum carthlicum, otherwise, the wheat sample to be detected is indicated not to carry the powdery mildew resistance gene PmDR803 of Triticum carthlicum.

4. The method according to claim 3, wherein the molecular marker primer in step (2) comprises a forward primer HENU629-F and a reverse primer HENU629-R, the nucleotide sequence of the forward primer HENU629-F is shown as SEQ ID NO:1, and the nucleotide sequence of the reverse primer HENU629-R is shown as SEQ ID NO:2.

5. The method according to claim 3, wherein a PCR amplification system in step (2) is 10 μL, comprising: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

6. The method according to claim 3, wherein a procedure of the PCR amplification in step (2) is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.
